(19) United States
(12) Patent Application Publication
(10) Pub. No.: US 2018/0346867 A1
(43) Pub. Date: Dec. 6, 2018

(54) Device For Reduced Oxygen Concentration Culture In Microfluidic Systems

(71) Applicant: PRESIDENT AND FELLOWS OF HARVARD COLLEGE, Cambridge, MA (US)

(72) Inventors: John W. K. OLIVER, Watertown, MA (US); Richard NOVAK, Jamaica Plain, MA (US); Michael J. CRONCE, Roxbury, MA (US); Donald E. INGBER, Boston, MA (US); Jeffrey C. WAY, Cambridge, MA (US)

(21) Appl. No.: 15/775,707

(22) PCT Filed: Nov. 14, 2016
(86) PCT No.: PCT/US2016/061883

§ 371 Date: May 11, 2018

Related U.S. Application Data
(60) Provisional application No. 62/348,850, filed on Jun. 10, 2016.
(60) Provisional application No. 62/339,579, filed on May 20, 2016.
(60) Provisional application No. 62/254,608, filed on Nov. 12, 2015.

(51) Int. Cl. C12M 1/34; C12M 3/06; C12M 1/12

(52) U.S. Cl. CPC C12M 41/34; C12M 25/02; C12M 23/16

(57) ABSTRACT

A low-oxygen system is directed to culturing a diverse microbiome and includes an anaerobic chamber and at least one microfluidic device removably inserted in the anaerobic chamber. The microfluidic device includes a first microchannel in which a first level of oxygen is maintained, and a second microchannel in which a second level of oxygen is maintained, the second level of oxygen having a greater oxygen concentration than the first level of oxygen. The microfluidic device further includes a membrane located at an interface region between the first microchannel and the second microchannel, the membrane further having a plurality of pores via which oxygen flows from the second microchannel to the first microchannel to form an oxygen gradient in the first microchannel. The system further includes a culture system provided in the first microchannel and including oxygen-sensitive anaerobic bacteria.

### CROSS-REFERENCE TO RELATED APPLICATIONS

This application claims priority to and benefit of U.S. Provisional Patent Application Ser. No. 62/254,608, filed on Nov. 12, 2015, U.S. Provisional Patent Application Ser. No. 62/339,579, filed on May 20, 2016, and U.S. Provisional Patent Application Ser. No. 62/348,850, filed on Jun. 10, 2016, each of which is hereby incorporated by reference herein in its entirety.

### GOVERNMENT SUPPORT

This invention was made with government support under grant no. HHSF223201310079C awarded by the U.S. Food and Drug Administration Agency (FDA) for “Organ-on-Chips Tools for Testing of Radiation Countermeasures,” and under grant no. HR011-15-C-0094 awarded by the Defense Advanced Research Projects Agency (DARPA) of the U.S. Department of Defense, Biological Robustness In Complex Settings (BRICS) for “Towards Re-Programming The Gut Microbiome.” The government has certain rights in the invention.

### FIELD OF THE INVENTION

The present invention relates generally to culturing tissues in a reduced oxygen atmosphere, and, more particularly, to a chamber for providing an oxygen gradient in cultures of hypoxic or anaerobic cells.

### BACKGROUND OF THE INVENTION

There is a need to culture tissues, including organs-on-chips, in a reduced oxygen atmosphere that more accurately reflects the in vivo microenvironments. This is beneficial for studying cell differentiation and normal tissue function in in-vivo conditions.

A further need is directed to culturing anaerobic microbiomes with human or other mammalian cells (e.g., intestinal tract with gut flora). Although hypoxic incubators and anaerobic chambers are available on the market, current incubators and chambers do not enable culture of mammalian cells with or without bacteria in an oxygen gradient experienced in vivo. For example, mammalian cells in the gut have adequate oxygen for metabolism, and extremely oxygen-sensitive bacteria can grow in the gut lumen under anaerobic conditions. This is an interplay of oxygen diffusion from the bloodstream (which is already at a lower oxygen partial pressure than that provided in standard incubators) and mammalian and bacterial cell metabolic oxygen consumption. The interplay of oxygen diffusion and the oxygen consumption results in a gradient of oxygen along the radius of the lumen and along the length of the digestive tract.

To culture a diverse array of bacteria, it is required to enable formation of microniches. Accordingly, one goal is to create microniches via stable oxygen gradients along the length of a microfluidic organ on chip and in a z-axis as a result of diffusion and/or metabolism.

Although several academic groups have developed oxygen gradient generators, these generators have not been applied to a microbiome culture. Furthermore, such devices are designed for gradient generation and are not widely applicable.

Thus, present embodiments are directed to solving the above and other needs. For example, some exemplary embodiments are directed to providing a more versatile hypoxic chamber that is suitable for a range of culture devices.

### SUMMARY OF THE INVENTION

According to one aspect of the present invention, a low-oxygen system is directed to culturing of a diverse microbiome. The low-oxygen system includes an anaerobic chamber, and at least one microfluidic device removably inserted in the anaerobic chamber. The microfluidic device includes a first microchannel in which a first level of oxygen is maintained, and a second microchannel in which a second level of oxygen is maintained. The second level of oxygen has a greater oxygen concentration than the first level of oxygen. The microfluidic device further includes a membrane located at an interface region between the first microchannel and the second microchannel, the membrane having a first surface facing the first microchannel and a second surface facing the second microchannel. The membrane further has a plurality of pores via which oxygen flows from the second microchannel to the first microchannel to form an oxygen gradient in the first microchannel. The low-oxygen system further includes a culture system provided in the first microchannel and adhered to the first surface of the membrane, the culture system including oxygen-sensitive anaerobic bacteria.

According to another aspect of the present invention, a low-oxygen system is directed to the culture of a diverse microbiome. The low-oxygen system includes an anaerobic chamber having an internal low-oxygen atmosphere and including a deoxygenated-medium reservoir, and an oxygenated-medium reservoir located external to the anaerobic chamber. The low-oxygen system further includes one or more microfluidic devices removably inserted in the anaerobic chamber, each of the microfluidic devices including an anaerobic microchannel in fluid communication with the deoxygenated-medium reservoir. The anaerobic microchannel includes oxygen in the form of an anaerobic oxygen gradient that ranges from a first anaerobic oxygen concentration to a second anaerobic oxygen concentration,

Each microfluidic device further includes an aerobic microchannel in fluid communication with the oxygenated-medium reservoir, the aerobic microchannel including oxygen in the form of an aerobic oxygen gradient that ranges from a first aerobic oxygen concentration to a second aerobic oxygen concentration. Each microfluidic device also includes a porous membrane located at an interface region between the anaerobic microchannel and the aerobic microchannel, oxygen diffusion occurring through pores of the membrane such that the anaerobic oxygen gradient is formed. The low-oxygen system also includes a culture system adhered to a surface of the porous membrane within the anaerobic microchannel, the culture system including oxygen-sensitive anaerobic bacteria.

According to yet another aspect of the present invention, a method is directed to culturing a diverse microbiome in a low-oxygen system. The low-oxygen system includes an anaerobic chamber containing a removable microfluidic device, the microfluidic device having a first microchannel separated by a porous membrane from a second microchannel. The method includes maintaining a first level of oxygen in the first microchannel, and maintaining a second level of oxygen in the second microchannel, the second level of oxygen having a greater oxygen concentration than the first level of oxygen. The method further includes diffusing oxygen from the second microchannel to the first microchannel to form an oxygen gradient, and culturing cells with an oxygen-sensitive anaerobic bacteria in the first microchannel.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1 is a top view of an organ-on-chip (“OOC”) device.

FIG. 2 is an enlarged cross-sectional front view representation along sectional lines 2-2 of FIG. 1.

FIG. 3 is an isometric view of an OOC device, according to an alternative embodiment.

FIG. 4 is a cross-sectional perspective front view representation along sectional lines 4-4 of FIG. 3.

FIG. 5 is a partial front view representation of the OOC device of FIG. 4.

FIG. 6A is a partial cutaway side view representation of the OOC device of FIG. 4 with a changing gradient along a Z-axis.

FIG. 6B is a partial cutaway side view representation of the OOC device of FIG. 4 with a uniform Z oxygen gradient along an X-axis and a changing gradient along the Z-axis.

FIG. 7 is a schematic representation of an anaerobic chamber.

FIG. 8 is an isometric view of an anaerobic chamber, according to one embodiment.

FIG. 9 is a front isometric view of an anaerobic chamber, according to another embodiment.

FIG. 10 is a back isometric view of the anaerobic chamber of FIG. 9.

FIG. 11. is a side isometric view of the anaerobic chamber of FIG. 9.

FIG. 12 is a cutaway isometric view of the anaerobic chamber of FIG. 11.

FIG. 13 is a timeline representative of oxygen level in a hypoxia farm pilot study.

FIG. 14A is a microscopic image illustrating villi-like structure growth at inlet in day one.

FIG. 14B is a microscopic image showing villi-like structure growth at inlet in day three.

FIG. 14C is a microscopic image showing villi-like structure growth at inlet in day five.

FIG. 14D is a microscopic image showing villi-like structure growth at inlet in day seven.

FIG. 15A is a microscopic image illustrating villi-like structure growth at outlet in day one.

FIG. 15B is a microscopic image showing villi-like structure growth at outlet in day three.

FIG. 15C is a microscopic image showing villi-like structure growth at outlet in day five.

FIG. 15D is a microscopic image showing villi-like structure growth at outlet in day seven.

FIG. 16A is another microscopic image showing villi-like structure growth at inlet in day seven.

FIG. 16B is another microscopic image showing villi-like structure growth at outlet in day seven.

FIG. 16C is another microscopic image showing villi-like structure growth at inlet in day seven.

FIG. 17A is a confocal microscopic image showing a perspective view of villi structures at inlet.

FIG. 17B is a confocal microscopic image showing a side view of villi structures at inlet.

FIG. 17C is a confocal microscopic image showing a perspective view of villi structures at outlet.

FIG. 17D is a confocal microscopic image showing a side view of villi structures at outlet.

FIG. 18 shows confocal sectioning of villi structures.

FIG. 19A shows carbonic anhydrase staining at inlet.

FIG. 19B shows carbonic anhydrase staining at outlet.

FIG. 20A is a bar chart illustrating colony forming units (“CFUs”) removed a number of days after inoculation from a first OOC device.

FIG. 20B is a bar chart illustrating CFUs removed a number of days after inoculation from a second OOC device.

FIG. 20C is a bar chart illustrating CFUs removed a number of days after inoculation from a third OOC device.

FIG. 20D is a bar chart illustrating CFUs removed a number of days after inoculation from a third OOC device.

FIG. 21A is a bar chart illustrating CFUs in both channels for the first OOC device of FIG. 20A.

FIG. 21B is a bar chart illustrating CFUs in both channels for the second OOC device of FIG. 20B.

FIG. 21C is a bar chart illustrating CFUs in both channels for the third OOC device of FIG. 20C.

FIG. 21D is a bar chart illustrating CFUs in both channels for the fourth OOC device of FIG. 20D.

FIG. 22 shows microscopic images representative of seeding in the OOC devices of FIGS. 20A-21D for three initial days.

FIG. 23 shows microscopic images representative of seeding in the OOC devices of FIGS. 20A-21D for three subsequent days.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.”

Generally, exemplary embodiments described below include 1) a chamber that permits hypoxic/anaerobic cell culture, as well as generation of oxygen gradients via diffusion-based oxygen delivery, and 2) a bioreactor or culture system that permits culture of a diverse microbiome, including oxygen-sensitive anaerobic bacteria. The chamber includes an enclosure configured to hold a plurality of organ-on-a-chip devices (e.g., four organ-on-a-chip devices). Each organ-on-a-chip device has one or more anaerobic channels, with each anaerobic channel being associated with a respective medium reservoir that is located inside the enclosure to maintain anaerobic conditions.

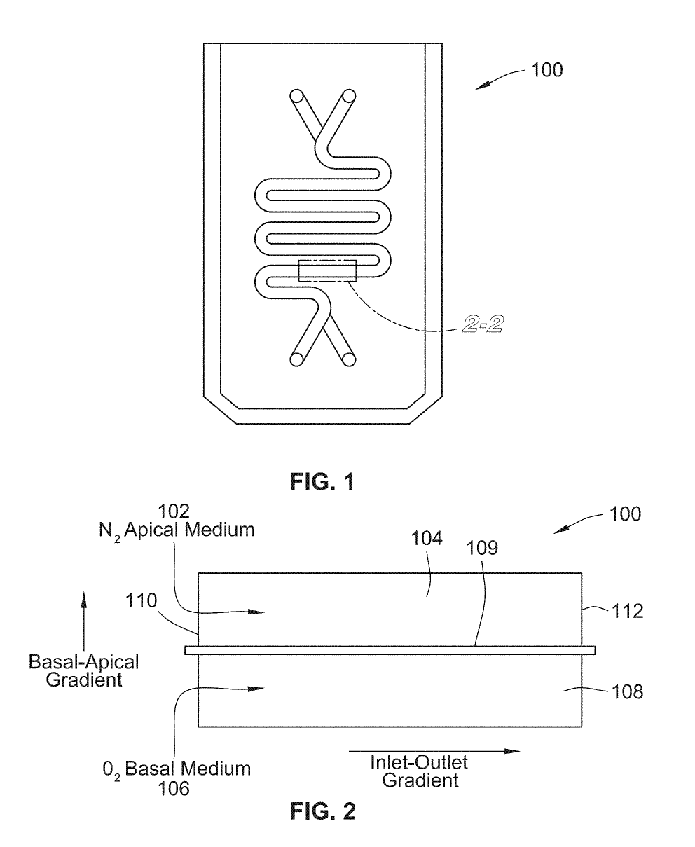

Referring to FIGS. 1 and 2, an OOC device 100 is an exemplary device intended for use in a hypoxic chamber setup, with nitrogen (N₂) flow, via water to humidify, into a chamber. The OOC device 100 is provided, for example, with a nitrogen apical medium 102 into a top compartment 104 and an oxygen (O₂) basal medium 106 into a bottom compartment 108. The mediums flow through the respective compartments 104, 108, which are separated at an interface region by a barrier 109, from an inlet 110 to an outlet 112. The apical medium 102 is in a hypoxic chamber, while the basal medium 106 is in an incubator atmosphere (as will be described in more detail below). In accordance with the provided configuration, gradients of oxygen are established (a) basal to apical and (b) inlet to outlet.

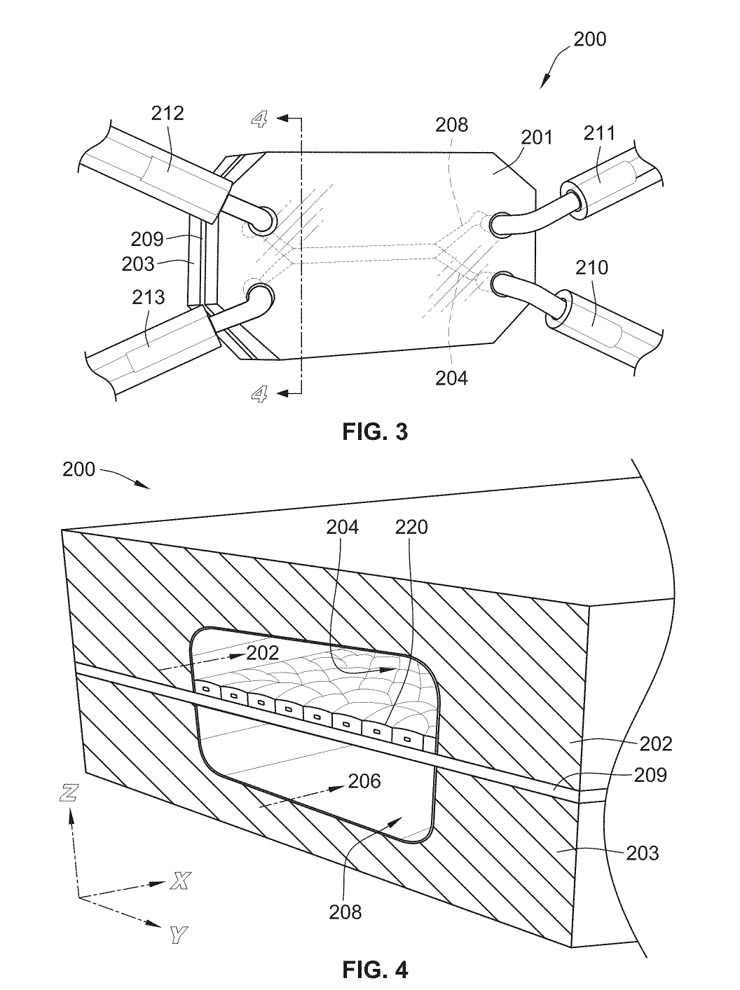

Referring to FIGS. 3 and 4, an OOC device 200 is typically made of a polymeric material including an upper body segment 201 and a lower body segment 203. The OOC device 200 has a first microchannel 204 and a second microchannel 208, through which respective mediums flow in accordance with desired experimental use. For example, as illustrated in FIG. 4 (and assuming that the first microchannel 204 is a top microchannel and the second microchannel 208 is a bottom microchannel), an apical medium 202 flows through the top microchannel 204 and a basal medium 206 flows through the bottom microchannel 208. For ease of understanding, the first microchannel 204 will be described below as being the top microchannel and the second microchannel 208 will be described as being the bottom microchannel. However, it is understood that, according to an alternative configuration, the first microchannel 204 is the bottom microchannel and the second microchannel 208 is the top microchannel.

The OOC device 200 further has a top fluid inlet 210 and a bottom fluid inlet 211 via which respective mediums are inserted into the respective microchannels 204, 208. The mediums exist from the respective microchannels 204, 208 via a top fluid outlet 212 and a bottom fluid outlet 213.

The OOC device 200 also has a barrier 209 that separates the microchannels 204, 208 at an interface region. The barrier 209 is optionally a semi-permeable barrier that permits migration of cells, particulates, media, proteins, and/or chemicals between the top microchannel 204 and the bottom microchannel 208. For example, the barrier 209 includes gels, layers of different tissue, arrays of micro-pillars, membranes, and combinations thereof. The barrier 209 optionally includes openings or pores to permit the migration of the cells, particulates, media, proteins, and/or chemicals between the top microchannel 204 and the bottom microchannel 208. According to one specific example, the barrier 209 is a porous membrane that includes a cell layer 220 (shown in FIG. 4) on at least one surface of the membrane.

According to alternative embodiments, the barrier 209 includes more than a single cell layer 220 disposed thereon. For example, the barrier 209 includes the cell layer 220 disposed within the top microchannel 204, the bottom microchannel 208, or each of the top and bottom microchannels 204, 208. Additionally or alternatively, the barrier 209 includes a first cell layer disposed within the top microchannel 208 and a second cell layer within the bottom microchannel 208. Additionally or alternatively, the barrier 209 includes a first cell layer and a second cell layer disposed within the top microchannel 204, the bottom microchannel 208, or each of the top and bottom microchannels 204, 208. Extracellular matrix gels are optionally used in addition to or instead of the cell layers.

Beneficially, the above-described various combinations provide for in-vitro modeling of various cells, tissues, and organs including three-dimensional structures and tissue-tissue interfaces such as brain astrocytes, kidney glomuralar epithelial cells, etc. In one embodiment of the OOC device 200, the top and bottom microchannels 204, 208 generally have a length of less than approximately 2 centimeters (“cm”), a height of less than approximately 200 microns (“μm”), and a width of less than approximately 400 μm. More details in reference to other features of the OOC device 200 are described, for example, in U.S. Pat. No. 8,647,861, titled “Organ Mimic Device With Microchannels And Methods Of Use And Manufacturing Thereof,” issued on Feb. 11, 2014, and which is incorporated by reference in its entirety.

The OOC device 200 is configured to simulate a biological function associated with cells, such as simulated organs, tissues, etc. One or more properties of a working medium, such as a fluid, may change as the working medium is passed through the microchannels 204, 208 of the OOC device 200, producing an effluent. As such, the effluent is still a part of the working medium, but its properties and/or constituents may change when passing through the OOC device 200.

The OOC device 200 optionally includes an optical window that permits viewing of the medium as it moves, for example, across the cell layer 220 and the barrier 209. Various image-gathering techniques, such as spectroscopy and microscopy, can be used to quantify and evaluate the medium flow or analyte flow through the cell layer 220.

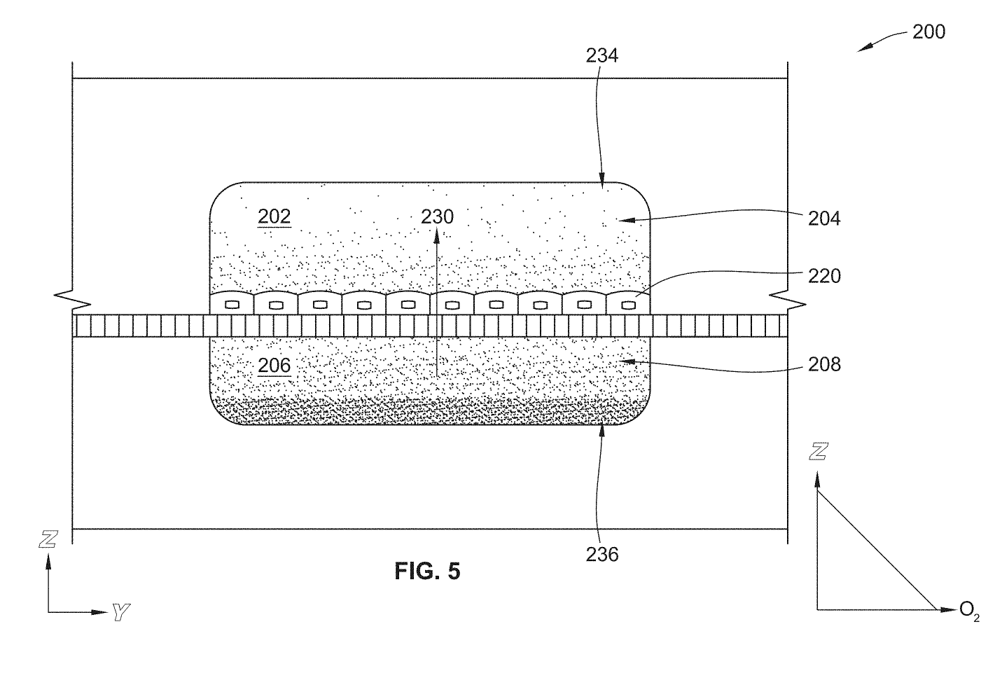

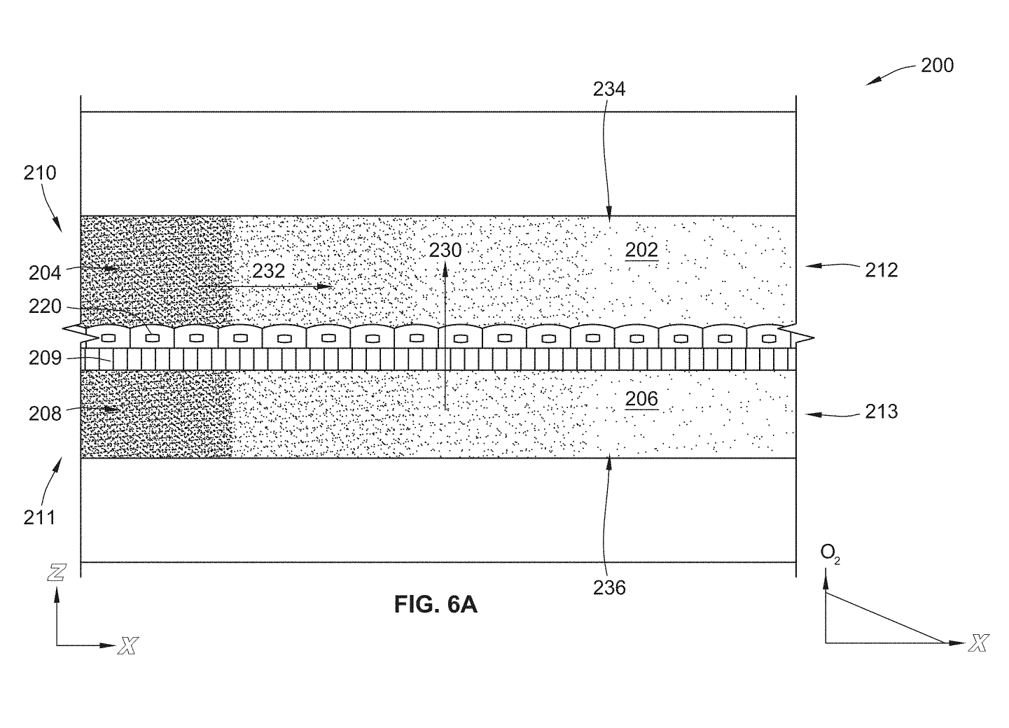

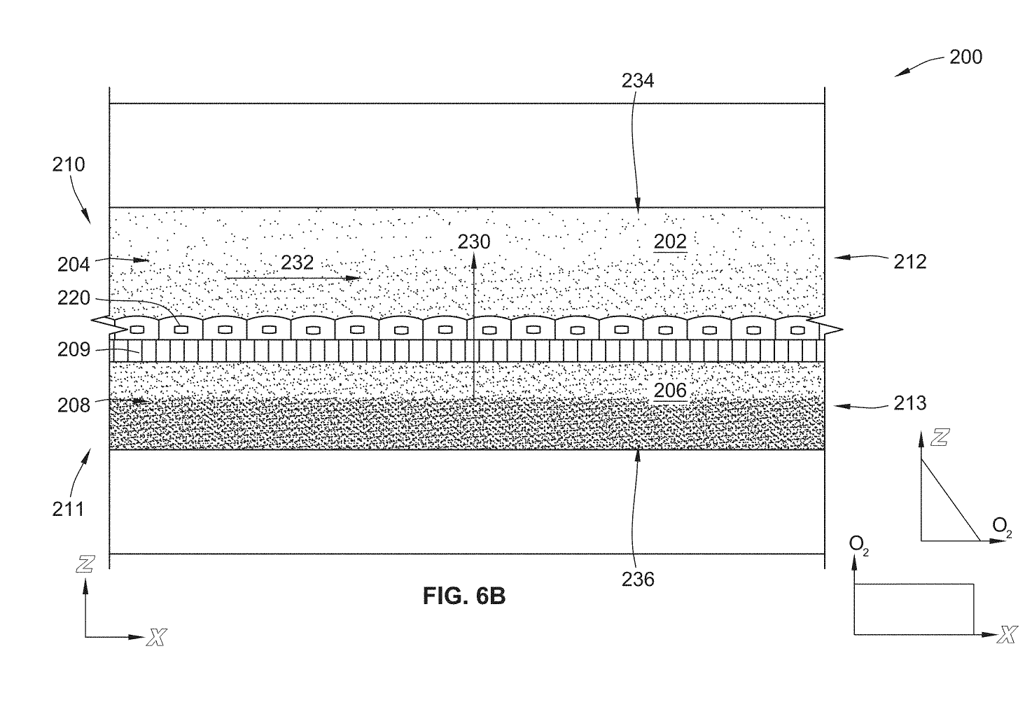

Referring generally to FIGS. 5, 6A, and 6B, the OOC device 200 is configured and maintained such that oxygen gradients 230, 232 are achieved in the microchannels 204, 208. For example, referring specifically to FIG. 5, a basal-apical gradient 230 is achieved along a Z-axis (e.g., microchannel height), with the level of oxygen decreasing towards an apex region 234 of the top microchannel 204. In other words, the level of oxygen is greater at a base region 236 of the bottom microchannel 208 than at the apex region 234 of the top microchannel 204.

In another example, referring specifically to FIG. 6A, an inlet-outlet gradient 232 is achieved along an X-axis (e.g., microchannel length), with the level of oxygen decreasing towards one or more of the microchannel fluid outlets 212, 213. In other words, the level of oxygen is greater at one or more of the microchannel fluid inlets 210, 211, than at one or more of the microchannel fluid outlets 212, 213.

Optionally, to achieve the basal-apical gradient 230 or the inlet-outlet gradient 232, the apical medium 202 that flows through the top microchannel 204 is nitrogen and the basal medium 206 that flows through the bottom microchannel 208 is oxygen. The levels of oxygen and nitrogen of the respective mediums, 202, 206 are provided and maintained at respective desired concentration levels.

In some embodiments, an oxygen gradient is established, enhanced or diminished by incorporating at least one oxygen permeable region. When such a region is in contact with a channel, it may allow oxygen to enter or exit the channel, for example by means of diffusion. Exemplary oxygen permeable regions can comprise, for example, semi-permeable membranes (e.g. porous PTFE membranes), materials with substantial oxygen permeability (e.g. PDMS), or thin sections (e.g. films) of materials with moderate to low intrinsic oxygen permeability. Without being bound by theory, the relative oxygen concentration or partial pressure difference between fluid present in the channel and one or more environments in contact with a said oxygen permeable region can determine the direction of oxygen transport. Accordingly, the device or at least one of the oxygen permeable regions may be disposed in contact with an anaerobic or hypoxic environment, for example, in order to enhance oxygen transport out of the channel. For example, at least a portion of the device may be placed within an anaerobic or hypoxic chamber. Conversely, by disposing device or at least one of the oxygen permeable regions in contact with an environment possessing a higher oxygen concentration or partial pressure relative to at least one region within the device or any fluid present within, one may drive oxygen into the device. Oxygen transport into or out of the device by means of the at least one oxygen permeable regions may be used to generate oxygen gradients along various axes (e.g. the basal-apical direction, longitudinal direction, or lateral direction), including along combinations of axes, or to produce complex gradient profiles (i.e. ones that aren't simply described by a single axis).

In a particular example, referring specifically to FIG. 6B, uniform oxygen diffusion is achieved along the X-axis, while an oxygen gradient is achieved along the Z-axis. In other words, in the illustrated embodiment, oxygen diffusion is uniform along a basal-apical direction 230 along the X-axis, while an oxygen gradient is achieved along an inlet-outlet direction 232 along the Z-axis. The X-axis uniformity and Z-axis gradient are achieved at least in part based on passive diffusion through a bottom area of the OOC device 200. Cut out of oxygen-blocking material below the OCC device 200 allows air to contact the permeable OOC device 200 only below the bottom microchannel 208, allowing for passive diffusion directly to the bottom microchannel 208 of the OOC device 200.

The passive diffusion of oxygen allows for the rate of oxygen diffusion to be decoupled from the rate of media perfusion through the bottom microchannel 208, allowing for separate control of oxygen and nutrient/shear stress properties of flow. Additionally, in some embodiments, growing epithelial cells or tightly associated aerobic bacteria (at a lower level in the oxygen gradient than anaerobic bacteria) require higher levels of oxygen than provided by flow alone. Accordingly, certain applications require uniform oxygen diffusion along the length of the OOC device 200 (e.g., along the X-axis) to increase the uniform surface area for testing. A small cutout (or channel) is used to allow diffusion of oxygen only in areas directly below the bottom of the microchannel 208. The cutout follows the design of the bottom microchannel 208 of the OOC device 200. According to another example, as illustrated in FIG. 1, an OOC device (e.g., OOC device 100) has a variable microchannel design. Oxygen diffusion is then dependent on the concentration of oxygen in the gas below the OOC device, which may be varied to control the oxygen gradient.

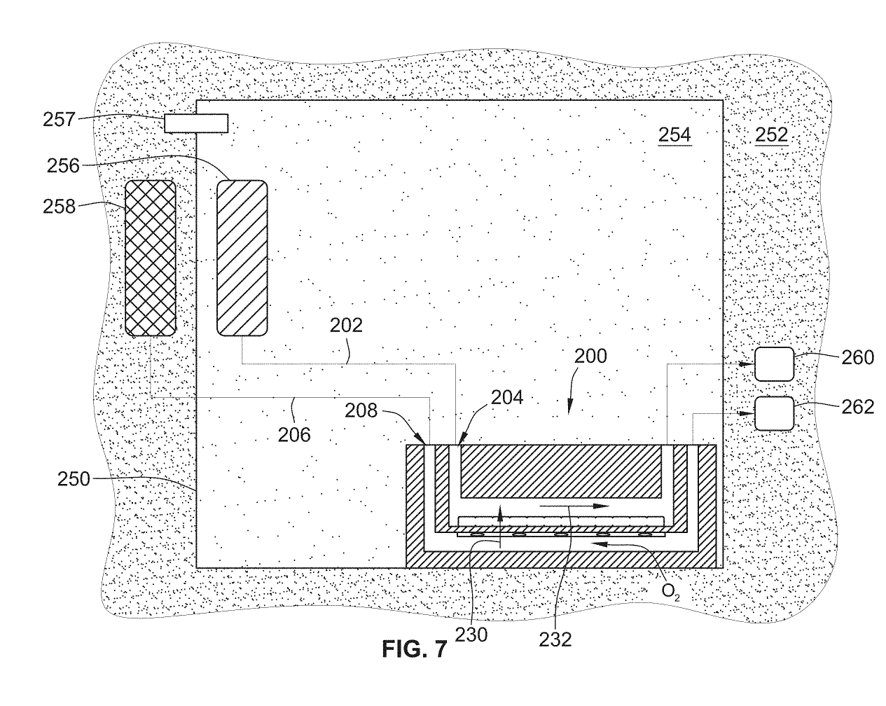

Referring to FIG. 7, an anaerobic chamber 250 is directed to providing a robust system for maintaining hypoxic conditions in a manner similar to in vivo microenvironments. For example, the anaerobic chamber 250 is configured to provide a potential large range of conditions for a varied mixture of bacteria to survive and/or grow, unlike a single container with one set of environmental parameters, including oxygen nutrients or shear rates. The anaerobic chamber 250 is further intended to provide a low-cost simple design for ease of sterilizing and fabrication, and to culture a variety of microfluidic and standard devices (such as the OOC device 200) with or without flow. The anaerobic chamber 250 is further amenable to frequent imaging of cultures without having to expose the cultures to ambient air, and is beneficial at least for culturing a microbiome ex vivo, for iteratively testing microbiome bacteria in unique niches, and for iteratively testing the interaction of microbiome cultures with intestinal epithelial cells.

The anaerobic chamber 250 is made from plastics, thermoplastics, thermoset polymers, rubber, acrylic, polycarbonate, metal, glass, and other materials, and has a structural enclosure that is positioned within an incubator at an incubator atmosphere 252. The incubator is a regular incubator or a hypoxic incubator that controls the maximum oxygen concentration. One or more OOC devices 200 are removably inserted within the anaerobic chamber 250 to achieve one or more of the oxygen gradients 230, 232. The anaerobic chamber 250 is maintained at a hypoxic or anoxic atmosphere 254 and includes a deoxygenated medium reservoir 256 that supplies, for example, the apical nitrogen medium 202 through the top microchannel 204.

To maintain hypoxic and/or anaerobic conditions inside the chamber 250, a gas line 257 is inserted that provides constant gas purging. According to some examples, the gas is nitrogen or a mixture of nitrogen with 5% of carbon dioxide (CO₂) to provide medium buffering. Other include a high-oxygen gas for testing for hyperoxia. To avoid desiccation, the gas 257 is passed through a liquid reservoir located in the enclosure. Tubing to and/or from the OOC devices is used to provide a constant perfusion of the tissue. Referring to aerobic channels, gas impermeable tubing is optionally used to avoid loss of oxygen through the tubing. The tubing is inserted with minimal contamination danger, and becomes sealed after the lid is placed on the device. Optionally, mating “capture” holes seal around tubing to minimize oxygen intake and to reduce purging gas use.

An oxygenated medium reservoir 258 is positioned external to the anaerobic chamber 250, to permit equilibration with the incubator atmosphere 252. The oxygenated medium reservoir 258 supplies, for example, the basal oxygen medium 206 through the bottom microchannel 208. After passing through the OOC device 200, the mediums 202, 206 are directed to respective pumps 260, 262 that are located external to the anaerobic chamber 250. Thus, according to this example, the chamber 250 is configured to remove oxygen from OOC devices 200 through a top surface, with an oxygen supply being pumped into the bottom microchannel 208. Alternatively, the oxygen supply is allowed to diffuse passively through a bottom surface of the OOC device 200.

According to one exemplary embodiment, the chamber 250 includes dual lids with a reduced lid size for allowing limited oxygen exposure to a medium. A larger lid seals tightly around the tubing, which leaves the enclosure and is not regularly removed, (especially not during regular medium changes and/or additions).

According to other exemplary embodiments, the chamber 250 includes one or more additional or optional features selected from the following examples. For example, the chamber 250 integrates sensors (e.g., oxygen, metabolite, electrical, optical, electrochemical, and protein) into the enclosure, and/or features for vacuum or mechanical stretch of OOC devices. In another example, the chamber 250 incorporates clamping and/or compression for hard to bond materials, such as membranes or biological materials. In yet another example, the chamber 250 is configured for bone marrow, neurons, stem cells, cartilage, and other tissues or cells where hypoxia is known to have a significant effect in a normal system; is configured to test ischemia or reperfusion injury or other diseases/injuries; and/or is configured to improve stem cell derived tissue maturation. In yet another example, the chamber 250 is configured to modulate oxygen gradients using flow rates in the microchannels of OOC devices, initial oxygen tension in the reservoirs/incubator, or by adding oxygen generating or scavenging compounds to the chamber 250 or to the culture systems (e.g., OCC devices). In yet another example, the chamber 250 integrates with fluid handling robots, imaging equipment (including a variety of microscopes and imaging modalities), and other gases such as sulfur dioxide (“SO₂”), carbon oxide (“CO”), radon, or nitrogen oxide (“NO”), for other applications and/or studies.

According to yet other exemplary embodiments, the chamber 250 is configured to study altitude sickness, to culture biopsies, and/or to culture and stabilize and/or validate fecal transplant donor samples. In other embodiments, the chamber 250 is configured to study, grow, and/or engineer microbial consortia, including, for example, natural microbiota, engineered consortia of select organisms, synthetically engineered pathways in bacteria, any combinations of bacteria, viruses, fungi, other protists, and parasites. In further embodiments, the chamber 250 is configured for use with high oxygen gas for testing hyperoxic conditions, and/or is configured for tissue engineering applications prior to implantation, e.g., build up vasculature, improve tissue maturity and/or function, and stress test. In further embodiments, oxygen gradients are extended to patterns and include gradients of other diffusible factors and/or gases. In other embodiments the chamber 250 is configured for culturing intestinal epithelial cells with anaerobic microbiome bacteria.

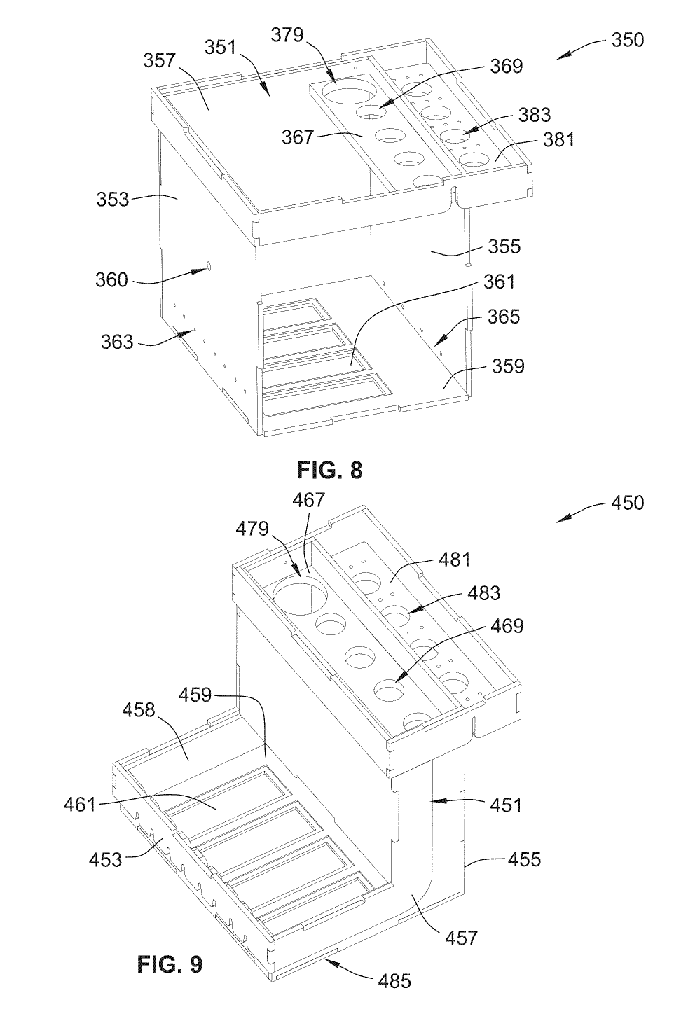

Referring to FIG. 8, according to one embodiment, the anaerobic chamber 250 is in the form of a generally rectangular chamber 350 that includes a structural enclosure 351 having a front wall 353, a rear wall 355, a left side wall 357, and a bottom wall 359. A top wall, generally parallel to the bottom wall 359 along a top surface of the chamber 350, and a right side wall, generally parallel to the left side wall along a right edge of the chamber 350 (which are not shown in FIG. 8 for clarity purposes), close-off the enclosure 351.

The chamber 350 is configured to accommodate one or more features described above in reference to the anaerobic chamber 250 and the OOC device 200, and is intended to hold a single OOC device 200, multiple OOC devices 200 (at least 12 would be readily feasible, but more are still achievable), or any other culture plate or container, including standard culture plates of various dimensions, TRANSWELL® insets, various reservoirs, bioreactors, and a range of microfluidic and mesofluidic devices. For example, the front wall 353 includes a vent 360 for venting excess gas in the chamber atmosphere, which is intended to be maintained at minimal oxygen levels. The bottom wall 359 separates the front wall 353 and the rear wall 355 along a bottom surface, and includes a plurality of slide insets 361 for accommodating respective OOC devices 200. To accommodate the flow of an oxygenated medium from an externally-located reservoir and the flow to an external pump, the front wall 353 also includes a plurality of outlet through-holes 363 and the rear wall 355 includes a plurality of inlet through-holes 365.

The chamber 350 further includes an internal tray 367 with anaerobic media holders 369 in the form of smaller circular apertures, and a bubbler holder 379 in the form of a relatively larger circular aperture. The bubbler holder 379 is intended for holding a bubbler that bubbles gas through liquid for humidifying dry gas. The internal tray 367 is mounted within the structural enclosure 351, adjacent to the rear wall 355 and proximate to the top surface of the anaerobic chamber 350. The chamber 350 also includes an external tray 381 with aerobic media holders 383 in the form of circular apertures. The external tray 381 is mounted external to the structural enclosure 351, adjacent to the rear wall 355 and proximate to the top surface of the anaerobic chamber 350.

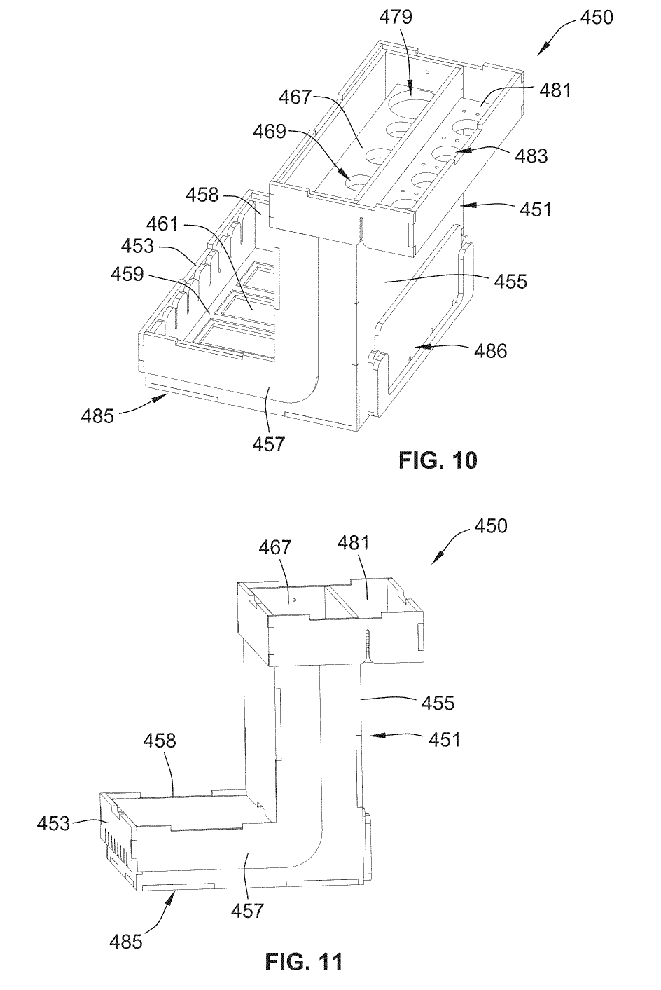

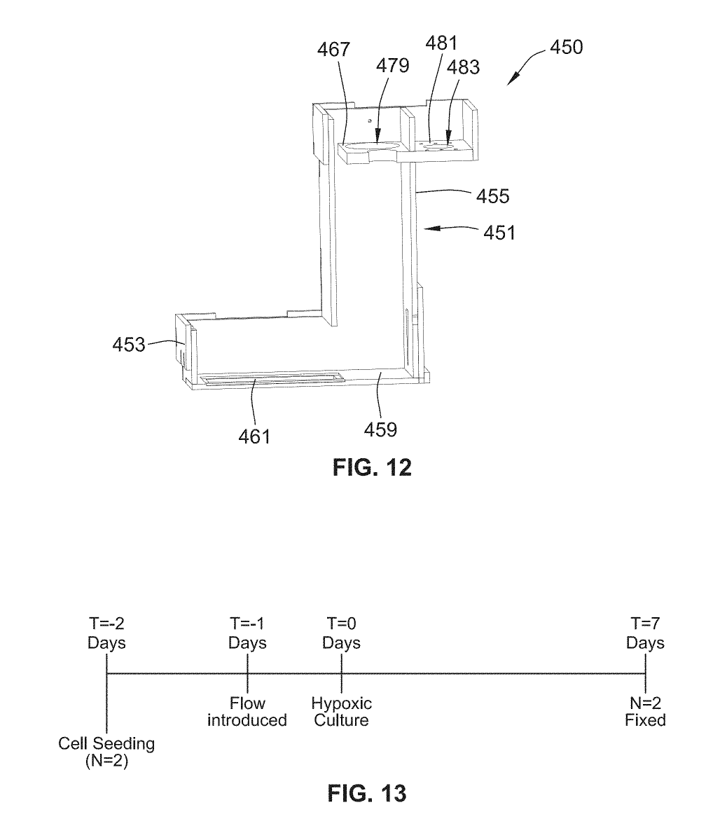

Referring to FIGS. 9-12, according to another embodiment, the anaerobic chamber 250 is in the form of a generally L-shaped chamber 450 for accommodating microscopy imaging and for facilitating ease of access. For example, the L-shape of the chamber 450 enables microscopy of cultured OOC devices 200 without having to expose the cultured OOC devices 200 to air. By way of example, the chamber has oxygen permeability that maintains hypoxic conditions for approximately 30 minutes without requiring a gas purge. Longer periods of microscopy can be achieved by reconnecting a purge gas line for time-lapse and other longer time scale imaging needs.

The chamber 450 includes a structural enclosure 451 having a low-profile front wall 453 that is connected to a high-profile rear wall 455 via two L-shaped side walls 457, 458. The structural enclosure 451 further has a bottom wall 459 with slide insets 461 for accommodating respective OOC devices 200, an internal tray 467 with anaerobic media holders 469 and a bubble holder 479, an external tray 481 with aerobic media holders 483, and comb tubing closures 485, 486. Optionally, the chamber 450 includes one or more additional features included in the anaerobic chamber 350 illustrated in FIG. 8.

Referring to FIG. 13, the experimental parameters of a hypoxia farm pilot study include a cell source of Caco2 BBE (P57) and human umbilical vein endothelial cells (“HUVECs”) (P7); apical media—DMEM-H, Glutamx, HEPES P/S, 10% FBS; basal media—Serum-reduced HUVEC medium (0.5%), P/S; a long channel stretchable OOC device format; and a no-stretch OOC device format. The study analytics include IF staining, with Carbonic Anhydrase II (Large intestines), ZO1 (tight junctions), and Villin (Villi/Small intestines). The oxygen level is maintained in accordance with the illustrated timeline, in which cell seeding occurs two days prior to the hypoxic culture, with flow being introduced one day prior to the hypoxic culture. The experiment is performed in a chamber similar or identical to any of the chambers 250, 350, 450 described above and using one or more OOC devices 250.

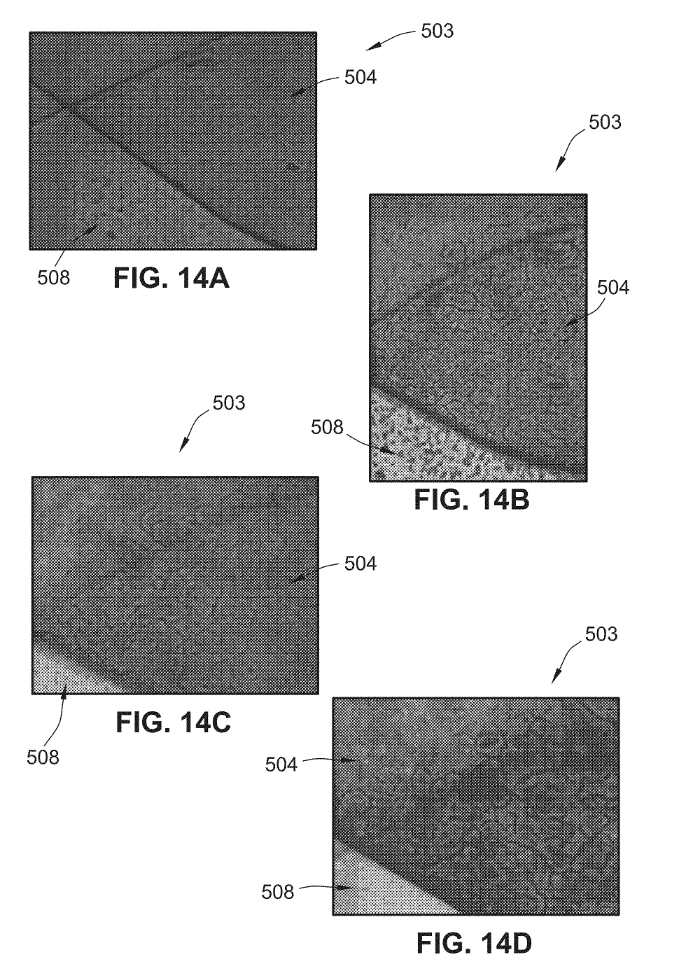

In Referring to FIGS. 14A-14D, brightfield images of growth show development of villi-like structures at an inlet area 503 over the course of the experiment described in reference to FIG. 13. The images show an anaerobic medium 504 region (e.g., a top microchannel of a an OOC device) and an oxygenated medium region 508 (e.g., a bottom microchannel of an OOC device). FIG. 14A shows an image taken on day one of the experiment, FIG. 14B shows an image taken on day three of the experiment, FIG. 14C shows an image taken on day five of the experiment, and FIG. 14D shows an image taken on day seven of the experiment. Of note, as shown in FIG. 14B, significant observable villi-like structures develop by day three of the experiment.

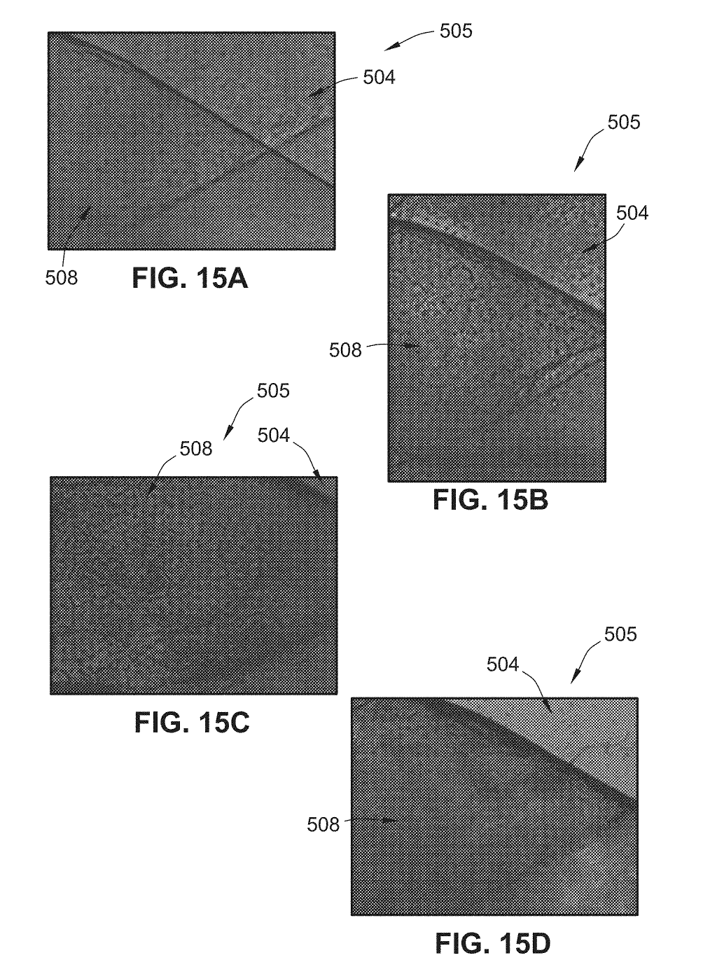

Referring to FIGS. 15A-15D, brightfield images of growth show the development of the villi-like structures at an outlet area 505 of the anaerobic and oxygenated medium regions 504, 508 over the course of the experiment described in reference to FIG. 13. FIG. 15A shows an image taken on day one of the experiment, FIG. 15B shows an image taken on day three of the experiment, FIG. 15C shows an image taken on day five of the experiment, and FIG. 15D shows an image taken on day seven of the experiment.

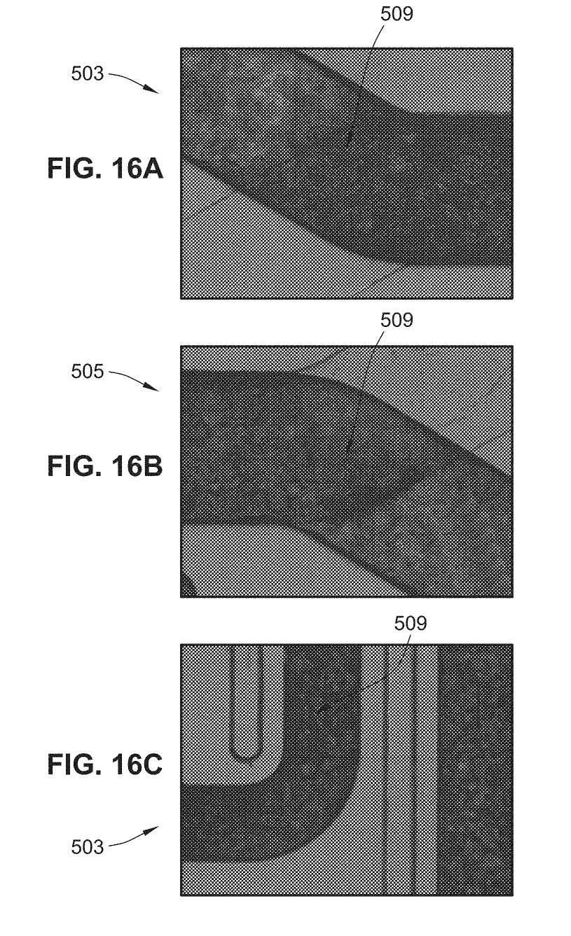

Referring to FIGS. 16A-16C, brightfield images show a growth comparison of villi-like structures as imaged in day 7 of the experiment. The comparison is between the inlet area 503 (FIGS. 16A and 16C) and the outlet area 505 (FIG. 16B). Notably, a thicker growth occurs at the inlet area 503, with a decreasing gradient of tissue height along a channel 509.

The villi-like structures correspond in height roughly proportionally to local oxygen concentrations, when tissue is exposed to a low oxygen gradient after a monolayer is formed and allowed to grow. Villi of up to 250 micrometers (“μm”) in height are achieved without stretch at the channel inlets 503 even after 3 days. Caco-2 cells survive throughout the chamber in a stable monolayer, even in an anaerobic channel inlet 503 and at the outlet 505 where there is expected to be minimal oxygen.

In another example, HUVECs can be cultured in an oxygenated channel (such as the bottom microchannel 208 of the OOC device 200) and can survive for an entire 7-day culture. These exemplary results indicate that a cultured OOC device 200, such as a gut-on-chip device, can be maintained in a chamber and can develop a range of villi heights and cell morphologies in a single OOC device 200.

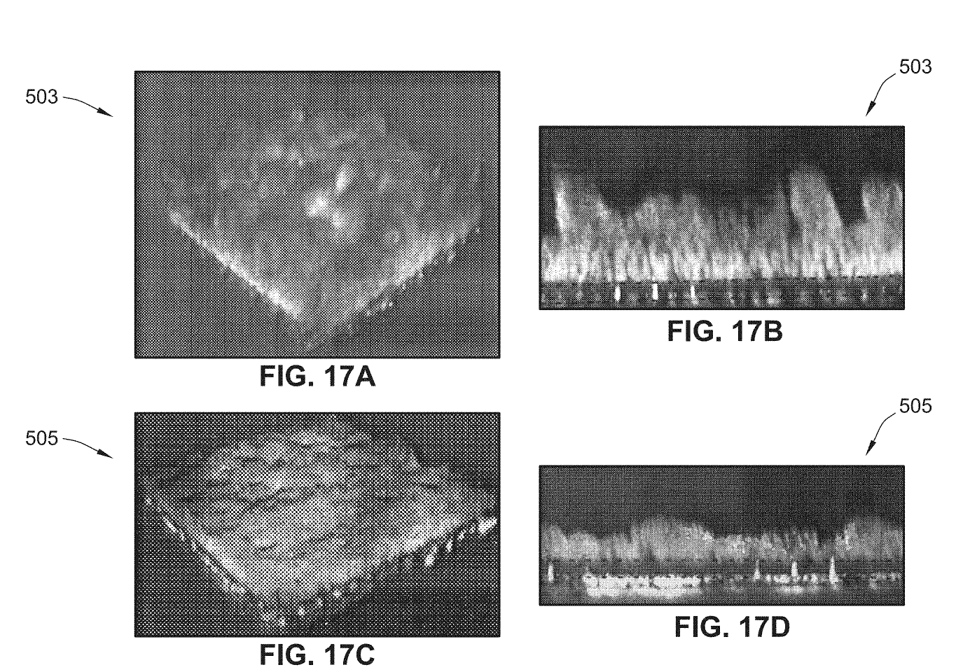

Referring to FIGS. 17A-17D, confocal images show the villi structures of the experiment described in reference to FIG. 13. Specifically, FIG. 17A shows a perspective image of the villi structures at the inlet area 503, FIG. 17B shows a side image of the villi structures at the inlet area 503, FIG. 17C shows a perspective image of the villi structures at the outlet area 505, and FIG. 17D shows a side image of the villis structures at the outlet area 505.

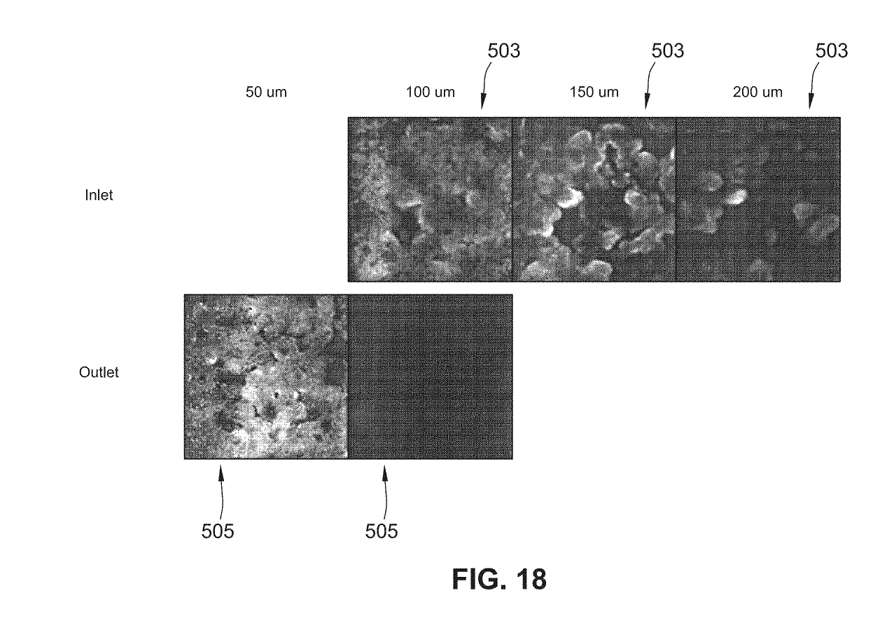

Referring to FIG. 18, confocal images show sectioning of the villi structures of the experiment described in reference to FIG. 13. Specifically, the images show sections of the villi structures at the inlet area 503, including sectioning at 100 μm, 150 μm, and 200 μm. The images further show sections of the villi structures at the outlet area 505, including sectioning at 50 μm and 100 μm.

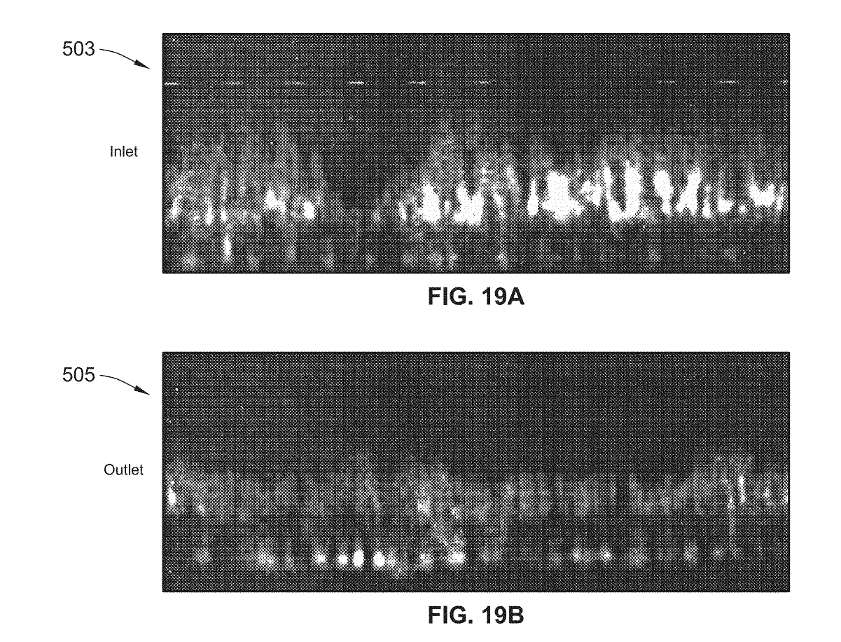

Referring to FIGS. 19A and 19B, images show carbonic anhydrase staining in the experiment described in reference to FIG. 13. Specifically, FIG. 19A shows an image at the inlet area 503 and FIG. 19B shows an image at the outlet 505.

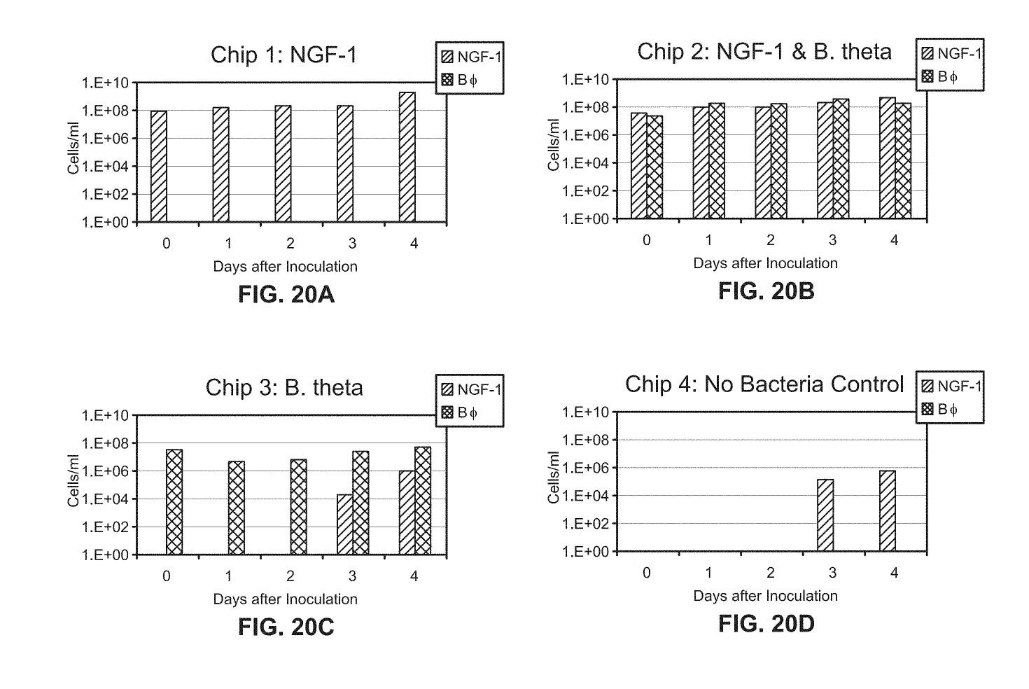

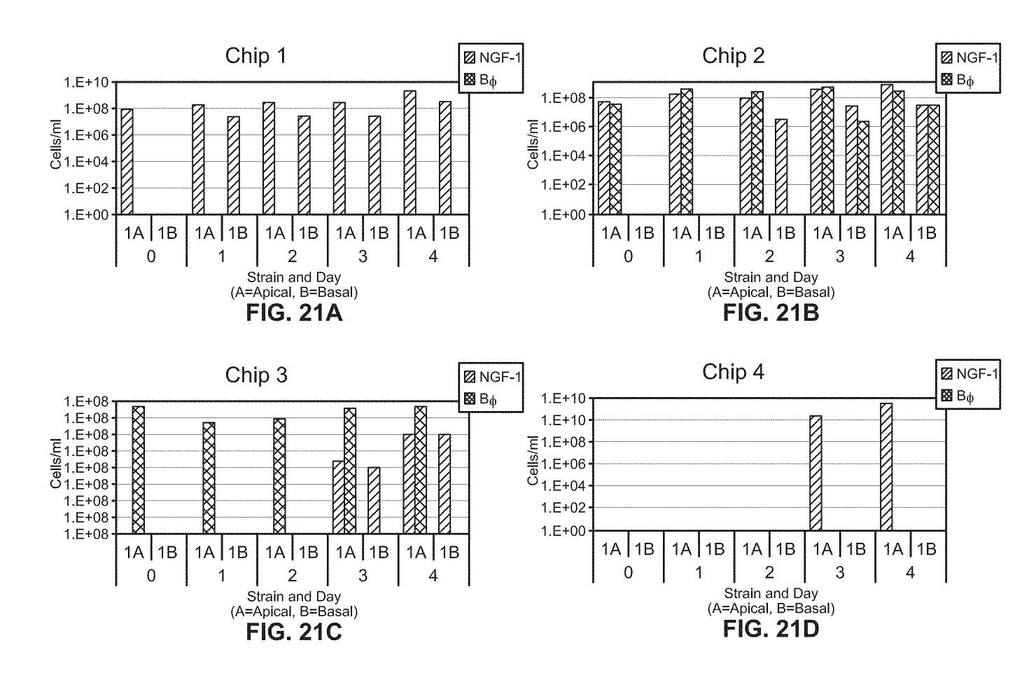

Referring to FIGS. 20A-20D and 21A-21D, bar charts illustrate experimental results for four different OOC devices, i.e., Chip 1, Chip 2, Chip 3, and Chip 4. Each of the OOC devices is subjected to different experimental conditions, as illustrated in each respective bar chart. Specifically, FIGS. 20A-20D show results in which CFUs are removed each day for at least up to four days after inoculation. In reference to FIGS. 21A-21D, show CFUs in both microchannels of the OOC devices. According to one embodiment, each of the OOC devices is similar to or identical to the OOC device 200 described above.

For example, an anaerobic apical lumen of the OOC devices is inoculated with Escherichia coli (“E. coli”), Bacteroides thetaiotaomicron (“B. theta”), or a co-culture of both species. B. theta is an anaerobic species that does not grow in aerobic conditions and provides proof of concept species for anaerobic culture. A stable culture of both species is achieved separately and together in this system for at least 4 days. Tissue barrier function, which is a measure of tissue stability, is maintained for most of the 4 days.

Optionally, the proposed model mimics oxygen diffusion gradients present in the human body. The model allows B. theta VPI-5482, which is an obligate anaerobe, to grow on top of intestinal epithelial cells without destruction of the epithelial cell layer. The proposed model includes commensal E. coli NGF-1 and co-cultures of both bacteria. All cultures grow, with B. theta alone generating less damage to the epithelial cell layer than E. coli alone. E. coli co-culture increases B. theta growth when using peristalsis.

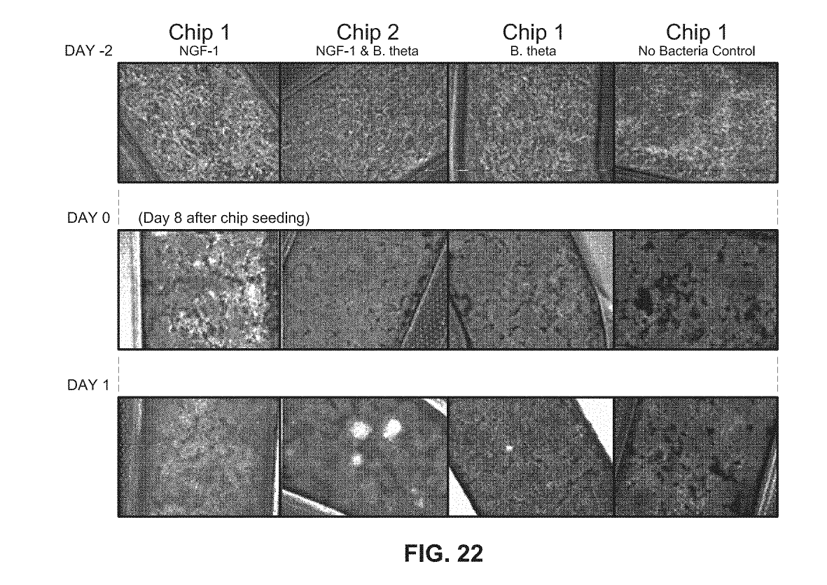

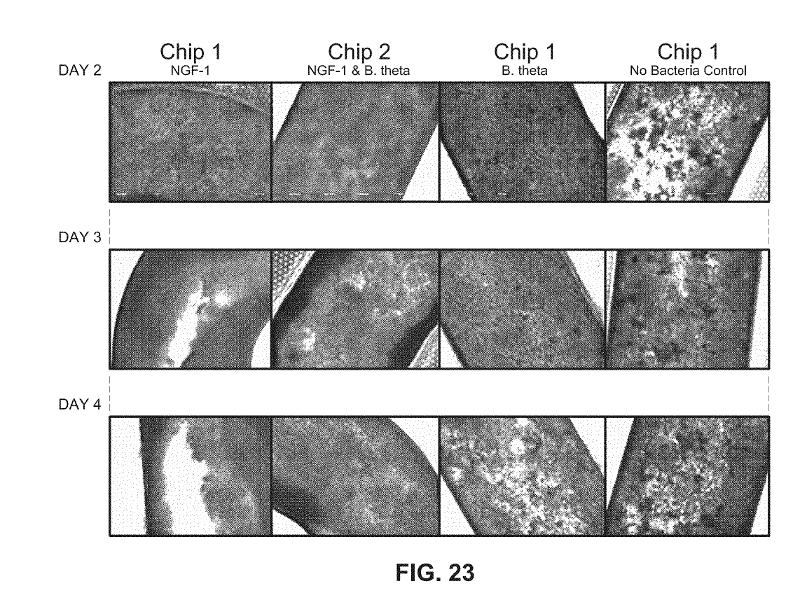

Referring to FIGS. 22 and 23, microscopic images show structure growth in each of the four OOC devices described above in reference to FIGS. 20A-20D and 21A-21D. The microscopic images refer to five different days of the experiment, including eight and nine days after seeding (shown in FIG. 22), and ten, eleven, and twelve days after seeding (shown in FIG. 23).

### Alternative Embodiments A1-A19

Alternative Embodiment A1. A method for perfusing a device with a low-oxygen fluid, comprising:

a) providing i) a fluid source and ii) a device having a first channel separated by a semi-permeable barrier from a second channel; and

b) perfusing either the first or second channel or both with a fluid containing a concentration of oxygen that is lower than the concentration of oxygen in the same fluid opened to ambient air.

Alternative Embodiment A2. The method of Alternative Embodiment A1, wherein the barrier includes a membrane.

Alternative Embodiment A3. The method of Alternative Embodiment A1, wherein the fluid is a gas.

Alternative Embodiment A4. The method of Alternative Embodiment A1, wherein the fluid includes tissue-culture medium.

Alternative Embodiment A5. The method of Alternative Embodiment A1, wherein the oxygen is dissolved in a liquid.

Alternative Embodiment A6. The method of Alternative Embodiment A1, wherein the fluid includes blood or a blood component.

Alternative Embodiment A7. The method of Alternative Embodiment A1, wherein the device is a microfluidic device and the first and second channels are first and second microchannels.

Alternative Embodiment A8. The method of Alternative Embodiment A1, further comprising c) culturing viable cells in the first or second channel.

Alternative Embodiment A9. The method of Alternative Embodiment A8, wherein the viable cells are animal cells.

Alternative Embodiment A10. The method of Alternative Embodiment A9, where the animal cells are co-cultured with bacteria.

Alternative Embodiment A11. The method of Alternative Embodiment A10, wherein the bacteria comprise anaerobic bacteria.

Alternative Embodiment A12. The method of Alternative Embodiment A10, wherein the animal cells are mammalian cells.

Alternative Embodiment A13. The method of Alternative Embodiment A9, wherein the cells are cultured on the barrier.

Alternative Embodiment A14. The method of Alternative Embodiment A1, wherein the anaerobic bacteria are oxygen-sensitive.

Alternative Embodiment A15. The method of Alternative Embodiment A1, wherein the fluid source includes a reservoir located internal to, and opened to, an anaerobic or hypoxic chamber.

Alternative Embodiment A16. The method of Alternative Embodiment A1, wherein the fluid source includes fluid that has been treated to reduce the concentration of oxygen.

Alternative Embodiment A17. The method of Alternative Embodiment A12, wherein the viable mammalian cells comprise cells from the intestinal tract.

Alternative Embodiment A18. The method of Alternative Embodiment A12, wherein the viable mammalian cells are Caco-2 cells.

Alternative Embodiment A19. The method of Alternative Embodiment A17, wherein the cells from the intestinal tract develop villi-like structures.

### Alternative Embodiments B1-B31

Alternative Embodiment B1. A method for perfusing a device with a fluid, comprising:

a) providing i) a fluid source and ii) a device having a first channel separated by a semi-permeable barrier from a second channel, each of the channels comprising an inlet and an outlet; and

b) perfusing either the first or second channel or both with a fluid containing a concentration of oxygen, wherein the concentration of oxygen is modified as the fluid flows through, such that a longitudinal gradient is formed whereby there is a different concentration of oxygen at one of the inlets than at the corresponding outlet.

Alternative Embodiment B2. The method of Alternative Embodiment B1, wherein the concentration of oxygen is reduced as the fluid flows through, such that a longitudinal gradient is formed whereby there is a greater concentration of oxygen at one of the inlets than at the corresponding outlet.

Alternative Embodiment B3. The method of Alternative Embodiment B1, wherein the concentration of oxygen is increased as the fluid flows through, such that a longitudinal gradient is formed whereby there is a lower concentration of oxygen at one of the inlets than at the corresponding outlet.

Alternative Embodiment B4. The method of Alternative Embodiment B1, wherein the concentration of oxygen in the fluid as the fluid is first introduced into the device is approximately the same as that in the same fluid opened to ambient air.

Alternative Embodiment B5. The method of Alternative Embodiment B1, wherein the concentration of oxygen in the fluid as the fluid is first introduced into the device is lower than the concentration of oxygen in the same fluid opened to ambient air.

Alternative Embodiment B6. The method of Alternative Embodiment B1, wherein the concentration of oxygen in the fluid as the fluid is first introduced into the device is greater than the concentration of oxygen in the same fluid opened to ambient air.

Alternative Embodiment B7. The method of Alternative Embodiment B1, wherein the barrier includes a membrane.

Alternative Embodiment B8. The method of Alternative Embodiment B1, wherein the fluid is a gas.

Alternative Embodiment B9. The method of Alternative Embodiment B1, wherein the fluid includes tissue-culture medium.

Alternative Embodiment B10. The method of Alternative Embodiment B1, wherein the oxygen is dissolved in a liquid.

Alternative Embodiment B11. The method of Alternative Embodiment B1, wherein the fluid includes blood or a blood component.

Alternative Embodiment B12. The method of Alternative Embodiment B1, wherein the device includes at least one oxygen permeable region that is contact with at least one of the first or second channels.

Alternative Embodiment B13. The method of Alternative Embodiment B12, wherein the oxygen permeable region includes an oxygen permeable material.

Alternative Embodiment B14. The method of Alternative Embodiment B1, wherein the device is in contact with an environment that is anaerobic or hypoxic.

Alternative Embodiment B15. The method of Alternative Embodiment B14, wherein at least one portion of the device is disposed within an anaerobic or hypoxic chamber.

Alternative Embodiment B16. The method of Alternative Embodiment B1, further comprising c) culturing viable cells in the first or second channel.

Alternative Embodiment B17. The method of Alternative Embodiment B16, wherein the device is a microfluidic device and the first and second channels are first and second microchannels.

Alternative Embodiment B18. The method of Alternative Embodiment B16, wherein the viable cells are animal cells.

Alternative Embodiment B19. The method of Alternative Embodiment B18, where the animal cells are co-cultured with bacteria.

Alternative Embodiment B20. The method of Alternative Embodiment B19, wherein the bacteria comprise anaerobic bacteria.

Alternative Embodiment B21. The method of Alternative Embodiment B18, wherein the animal cells are mammalian cells.

Alternative Embodiment B22. The method of Alternative Embodiment B16, wherein the cells are cultured on the barrier.

Alternative Embodiment B23. The method of Alternative Embodiment B20, wherein the anaerobic bacteria are oxygen-sensitive.

Alternative Embodiment B24. The method of Alternative Embodiment B21, wherein the viable mammalian cells comprise cells from the intestinal tract.

Alternative Embodiment B25. The method of Alternative Embodiment B21, wherein the viable mammalian cells are Caco-2 cells.

Alternative Embodiment B26. The method of Alternative Embodiment B24, wherein the cells from the intestinal tract develop villi-like structures.

Alternative Embodiment B27. The method of Alternative Embodiment B21, wherein the viable mammalian cells comprise primary enterocytes.

Alternative Embodiment B28. The method of Alternative Embodiment B21, wherein the viable mammalian cells are selected to mimic one or more functions of at least one portion of the intestinal tract.

Alternative Embodiment B29. The method of Alternative Embodiment B1, wherein the fluid source includes a reservoir located internal to, and opened to, an anaerobic or hypoxic chamber.

Alternative Embodiment B30. The method of Alternative Embodiment B1, wherein the fluid source includes fluid that has been treated to reduce the concentration of oxygen contained within it.

Alternative Embodiment B31. The method of Alternative Embodiment B1, wherein both microfluidic channels are perfused with the same fluid such that a longitudinal gradient is created in both channels.

### Alternative Embodiments C1-C21

Alternative Embodiment C1. A method for perfusing a device with a fluid, comprising:

a) providing i) first and second fluid sources, the first source comprising a first fluid containing a concentration of oxygen, the second fluid source comprising a second fluid containing a concentration of oxygen that is higher than the concentration in the first fluid; and ii) a device having a first channel separated by a porous membrane from, and positioned above, a second channel; and

b) perfusing the first channel with the first fluid and the second channel with the second fluid such that a vertical gradient is formed whereby there is a greater concentration of oxygen in the second channel as compared to that of first channel.

Alternative Embodiment C2. The method of Alternative Embodiment C1, wherein the concentration of oxygen in the first fluid is lower than the concentration of oxygen in the same fluid opened to ambient air.

Alternative Embodiment C3. The method of Alternative Embodiment C1, wherein the barrier includes a membrane.

Alternative Embodiment C4. The method of Alternative Embodiment C1, wherein the fluid is a gas.

Alternative Embodiment C5. The method of Alternative Embodiment C1, wherein the fluid includes tissue-culture medium.

Alternative Embodiment C6. The method of Alternative Embodiment C1, Wherein the oxygen is dissolved in a liquid.

Alternative Embodiment C7. The method of Alternative Embodiment C1, wherein the fluid includes blood or a blood component.

Alternative Embodiment C8. The method of Alternative Embodiment C1, wherein the device is a microfluidic device and the first and second channels are first and second microchannels.

Alternative Embodiment C9. The method of Alternative Embodiment C1, further comprising c) culturing viable cells in the first or second channel.

Alternative Embodiment C10. The method of Alternative Embodiment C9, wherein the viable cells are animal cells.

Alternative Embodiment C11. The method of Alternative Embodiment C10, where the animal cells are co-cultured with bacteria.

Alternative Embodiment C12. The method of Alternative Embodiment C11, wherein the bacteria comprise anaerobic bacteria.

Alternative Embodiment C13. The method of Alternative Embodiment C9, wherein the animal cells are mammalian cells.

Alternative Embodiment C14. The method of Alternative Embodiment C9, wherein the cells are cultured on the barrier.

Alternative Embodiment C15. The method of Alternative Embodiment C12, wherein the anaerobic bacteria are oxygen-sensitive.

Alternative Embodiment C16. The method of Alternative Embodiment C1, wherein the fluid source includes a reservoir located internal to, and opened to, an anaerobic or hypoxic chamber.

Alternative Embodiment C17. The method of Alternative Embodiment C1, wherein the first fluid source includes fluid that has been treated to reduce the concentration of oxygen.

Alternative Embodiment C18. The method of Alternative Embodiment C13, wherein the viable mammalian cells comprise cells from the intestinal tract.

Alternative Embodiment C19. The method of Alternative Embodiment C13, wherein the viable mammalian cells are Caco-2 cells.

Alternative Embodiment C20. The method of Alternative Embodiment C17, wherein the cells from the intestinal tract develop villi-like structures.

Alternative Embodiment C21. The method of Alternative Embodiment C16, wherein the second fluid source includes a reservoir located external to an anaerobic chamber.

### Alternative Embodiments D1-D3

Alternative Embodiment D1. A method for perfusing a fluidic device with a fluid, comprising:

a) providing i) first and second fluid sources, the first source comprising a first fluid containing a concentration of oxygen, the second fluid source comprising a second fluid having a partial pressure of oxygen that is higher than the partial pressure of the first fluid; and ii) a device having a first channel separated by a semi-permeable barrier from, and positioned above, a second channel; and

b) perfusing the first channel with the first fluid and the second channel with the second fluid such that a vertical gradient is formed whereby there is a greater concentration of oxygen in the second channel as compared to that of first channel.

Alternative Embodiment D2. The method of Alternative Embodiment D1, wherein the barrier includes a membrane.

Alternative Embodiment D3. The method of Alternative Embodiment D1, wherein the device is a microfluidic device with microfluidic first and second channels.

Each of these embodiments and obvious variations thereof is contemplated as falling within the spirit and scope of the claimed invention, which is set forth in the following claims. Moreover, the present concepts expressly include any and all combinations and subcombinations of the preceding elements and aspects.

1-15. (canceled)

16. A method for culturing in a low-oxygen system, the low-oxygen system including an anaerobic chamber containing a removable microfluidic device, the microfluidic device having a first microchannel separated by a porous membrane from a second microchannel, the method comprising:
maintaining a first level of oxygen in the first microchannel;
maintaining a second level of oxygen in the second microchannel, the second level of oxygen having a greater oxygen concentration than the first level of oxygen;
diffusing oxygen from the second microchannel to the first microchannel to form an oxygen gradient; and
culturing epithelial cells with an oxygen-sensitive anaerobic bacteria in the first microchannel, wherein said culturing comprises growing said anaerobic bacteria on top of a layer of said epithelial cells without destruction of the epithelial layer.

17. The method of claim 16, further comprising forming the oxygen gradient longitudinally across a length of the first microchannel.

18. The method of claim 16, further comprising forming the oxygen gradient transversely across a height of the first microchannel.

19. The method of claim 16, further comprising flowing an oxygenated medium into the second microchannel from an oxygenated-medium reservoir located external to the anaerobic chamber.

20. The method of claim 16, further comprising flowing a deoxygenated medium into the first microchannel from a deoxygenated-medium reservoir located within the anaerobic chamber.

21-94. (canceled)
